(19) United States
(12) Patent Application Publication
(10) Pub. No.: US 2021/0030737 A1
(43) Pub. Date: Feb. 4, 2021

(54) Triple Negative Breast Cancer Treatment Method

(71) Applicant: EXELIXIS, INC., Alameda, CA (US)

(72) Inventors: Sara M. TOLANEY, Brookline, MA (US); Dan G. DUDA, Charlestown, MA (US)

(73) Assignee: Exelixis, Inc., Alameda, CA (US)

(21) Appl. No.: 16/095,221

(22) PCT Filed: Apr. 18, 2017
(86) PCT No.: PCT/US2017/028129

§ 371 Date: Oct. 19, 2018

Related U.S. Application Data
(60) Provisional application No. 62/324,711, filed on Apr. 19, 2016.

(51) Int. Cl. A61K 31/47; A61K 47/38; A61K 47/26; A61K 47/02; A61K 47/12; A61K 9/28; A61K 38/17; A61K 39/395; A61P 35/00

(52) U.S. Cl. CPC A61K 31/47; A61K 47/38; A61K 47/26; A61K 47/02; A61P 35/00; A61K 9/2813; A61K 9/2866; A61K 38/1774; A61K 39/3955; A61K 47/12

(57) ABSTRACT

Disclosed is a method of treating triple negative breast cancer in a human patient, comprising administering to the patient an amount of cabozantinib or a pharmaceutically acceptable salt thereof, wherein the amount of cabozantinib is sufficient to activate the immune system.

### RELATED APPLICATIONS

This application claims priority to U.S. Application Ser. No. 62/324,711, filed Apr. 19, 2017. The entire contents of the aforementioned application are incorporated herein by reference.

### TECHNICAL FIELD

Disclosed is a method for treating triple negative breast cancer. The method employs cabozantinib or cabozantinib in combination with other therapies or agents.

### BACKGROUND

Breast cancer is the second highest cause of cancer mortality among American women. Triple-negative breast cancer (TNBC) refers to any breast cancer that does not express the genes for estrogen receptor (ER), progesterone receptor (PR), or Her2/neu. TNBC accounts for 15-25% of breast cancers. It is more difficult to treat than other breast cancer subtypes because most chemotherapies target one of the three receptors. TNBC has a relapse pattern that is very different from hormone-positive breast cancers. The risk of relapse is much higher for the first 3-5 years but drops sharply and substantially below that of hormone-positive breast cancers after that. This relapse pattern has been recognized for all types of triple-negative cancers for which sufficient data exists, although the absolute relapse and survival rates differ across subtypes.

While triple-negative breast cancer (TNBC) represents only 15-25% of breast cancers, it is associated with high-grade disease, early visceral metastases, and death.

Thus, there is an urgent need for effective targeted therapeutics to treat TNBC. Currently, there are no targeted therapies for this subtype.

As a result, a need remains for new therapies to treat TNBC.

### SUMMARY

These and other needs are met by the present invention, which is directed to a method of treating TNBC in human patients. The method employs cabozantinib. The invention is also directed to the use of cabozantinib for treating TNBC in human patients. The invention is also directed to the use of cabozantinib in the manufacture of a medicament for treating TNBC in human patients.

The methods and associated uses disclosed herein employ cabozantinib, which is an oral inhibitor of tyrosine kinases including MET, VEGF receptors, and AXL. Cabozantinib has the structure depicted below.

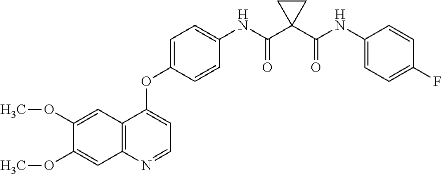 [SMILES: COc1cc2nccc(Oc3ccc(NC(=O)C4(C(=O)Nc5ccc(F)cc5)CC4)cc3)c2cc1OC]

In preferred embodiments, the (S)-malate salt of cabozantinib is administered. Cabozantinib (S)-malate is described chemically as N-(4-(6,7-dimethoxyquinolin-4-yloxy)phenyl)-N′-(4-fluorophenyl)cyclopropane-1,1-dicarboxamide, (2S)-hydroxybutanedioate. The molecular formula is C₂₈H₂₄FN₃O₅.C₄H₆O₅, and the molecular weight is 635.6 Daltons as malate salt. The chemical structure of cabozantinib (S)-malate salt is depicted below.

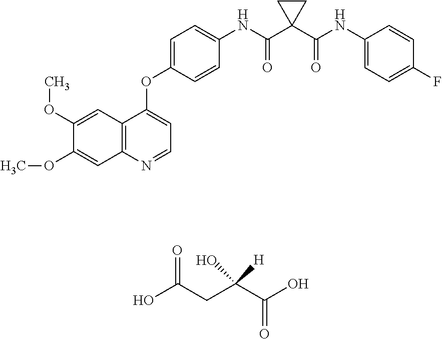 [SMILES: COc1cc2nccc(Oc3ccc(NC(=O)C4(C(=O)Nc5ccc(F)cc5)CC4)cc3)c2cc1OC ; O=C(O)C[C@H](O)C(=O)O]

Cabozantinib (S)-malate as a capsule formulation (COMETRIQ®) has been approved for the treatment of medullary thyroid cancer. Cabozantinib (S)-malate as a tablet formulation (CABOMETYX®) has been approved for the treatment of advanced renal cell carcinoma in patients who have received prior antio-angiogenic therapy.

Cabozantinib is an inhibitor of MET, a receptor tyrosine kinase that promotes cell proliferation, invasion, and survival when activated by its ligand, hepatocyte growth factor (HGF). MET and HGF overexpression are associated with tumor hypoxia, increased invasiveness and metastasis, and reduced survival in metastatic breast cancer. Furthermore, MET expression is disproportionately elevated in TNBC and associated with poorer prognosis. MET copy number was found to be elevated in 14% of TNBC, as opposed to 8% of hormone receptor-positive (HR1) breast cancer, and 7% of human epidermal growth receptor 2-positive (HER21) breast cancer. Preclinical studies suggest that MET expression drives differentiation of tumors into the TNBC subtype. Mice harboring an activating mutant MET knock-in or mutant MET transgene under mouse mammary tumor virus promoter developed TNBCs, suggesting that inhibition of MET signaling may be a promising therapeutic approach.

In one aspect, the invention is directed to a method of treating triple negative breast cancer in a human patient, comprising administering to the patient an amount of cabozantinib or a pharmaceutically acceptable salt thereof, wherein the amount of cabozantinib is sufficient to activate the immune system. In this and other aspects, the cabozantinib is administered as cabozantinib (S)-malate.

In another aspect, the invention is directed to a method of treating triple negative breast cancer in a human patient, comprising administering to a patient in need of such treatment cabozantinib or a pharmaceutically acceptable salt thereof at a dose which activates circulating cell biomarkers.

This and other aspects and embodiments is described herein below.

### BRIEF DESCRIPTION OF THE FIGURES

FIG. 1 depicts the experimental design for the study.

FIG. 2A depicts a waterfall plot of best response.

FIG. 2B and FIG. 2C depict the probability of progression free survival over time.

FIG. 3A, FIG. 3B, and FIG. 3C summarize changes in circulating tumor biomarkers over the course of the study.

### DETAILED DESCRIPTION

As indicated above, the invention is directed to a method of treating triple negative breast cancer in a human patient, comprising administering to the patient an amount of cabozantinib or a pharmaceutically acceptable salt thereof, wherein the amount of cabozantinib is sufficient to activate the immune system.

In one embodiment, the cabozantinib is administered as cabozantinib (S)-malate.

In a further embodiment, the cabozantinib (S)-malate is administered as a tablet formulation comprising approximately (% w/w):
30-32 percent by weight of cabozantinib, (S)-malate salt;
38-40 percent by weight of microcrystalline cellulose;
18-22 percent by weight of lactose;
2-4 percent by weight of hydroxypropyl cellulose;
4-8 percent by weight of croscarmellose sodium;
0.2-0.6 percent by weight of colloidal silicon dioxide;
0.5-1 percent by weight of magnesium stearate; and further comprising:
a film coating material comprising hypromellose, titanium dioxide, triacetin, and iron oxide yellow.

In a further embodiment, the cabozantinib (S)-malate is administered as a tablet formulation comprising approximately (% w/w):
31-32 percent by weight of cabozantinib, (S)-malate salt;
39-40 percent by weight of microcrystalline cellulose;
19-20 percent by weight of lactose;
2.5-3.5 percent by weight of hydroxypropyl cellulose;
5.5-6.5 percent by weight of croscarmellose sodium;
0.25-0.35 percent by weight of colloidal silicon dioxide;
0.7-0.8 percent by weight of magnesium stearate; and further comprising:
3.9-4.1 percent by weight of a film coating material comprising hypromellose, titanium dioxide, triacetin, and iron oxide yellow.

In a further embodiment, the cabozantinib (S)-malate is administered as a tablet formulation containing 20, 40, or 60 mg of cabozantinib free base equivalent (FBE).

In a further embodiment, the cabozantinib (S)-malate is administered as a tablet formulation selected from the group consisting of:

| | Theoretical Quantity (mg/unit dose) | | |
|---|---|---|---|
| Ingredient | 20-mg Tablet* | 40-mg Tablet* | 60-mg Tablet* |
| Cabozantinib (S)-malate | 25.34 | 50.69 | 76.03 |
| Microcrystalline Cellulose, PH-102 | 31.08 | 62.16 | 93.24 |
| Lactose Anhydrous, 60M | 15.54 | 31.07 | 46.61 |
| Hydroxypropyl Cellulose, EXF | 2.400 | 4.800 | 7.200 |
| Croscarmellose Sodium | 4.800 | 9.600 | 14.40 |
| Colloidal Silicon Dioxide | 0.2400 | 0.4800 | 0.7200 |
| Magnesium Stearate (Non-Bovine) | 0.6000 | 1.200 | 1.800 |
| Opadry ® Yellow (03K92254) | 3.200 | 6.400 | 9.600 |
| Total tablet weight | 83.20 | 166.4 | 249.6 |

*Free Base Equivalent (FBE)

In a further embodiment, the cabozantinib (S)-malate is administered once daily.

In a further embodiment, the amount of cabozantinib that is administered once daily is 60 mg FBE.

In a further embodiment, the amount of cabozantinib administered is sufficient to activate the immune system of a patient, increasing the number of circulating CD3+ cells. In another embodiment, the number of CD8+ T cells is increased. In another embodiment, the number of CD4+ cells is increased. In another embodiment, the number of CD56+ NK cells is increased. In another embodiment, the number of CD+14 monocytes in the patient is decreased.

In a further embodiment, the amount of cabozantinib administered is sufficient to activate the immune system of a patient, increasing the number of circulating CD3+ cells and CD8+ T cells. In a further embodiment, the amount of cabozantinib administered is sufficient to activate the immune system of a patient, increasing the number of circulating CD3+ cells, CD8+ T cells, and CD4+ cells. In a further embodiment, the amount of cabozantinib administered is sufficient to activate the immune system of a patient, increasing the number of circulating CD3+ cells, CD8+ T cells, CD4+ cells, and CD56+NK cells. In another embodiment, the number of circulating CD3+ cells and CD8+ T cells is increased, and the number of CD+14 monocytes in the patient is decreased. In another embodiment, the number of circulating CD3+ cells, CD8+ T, and CD4+ cells is increased, and the number of CD+14 monocytes in the patient is decreased.

In another embodiment, the number of circulating CD3+ cells, CD8+ T, CD4+ cells, and CD56+NK cells is increased, and the number of CD+14 monocytes in the patient is decreased.

In another aspect, the invention is directed to a method of treating triple negative breast cancer in a human patient, comprising administering to a patient in need of such treatment cabozantinib or a pharmaceutically acceptable salt thereof at a dose which activates circulating cell biomarkers.

In one embodiment of this aspect, circulating cell biomarker activation is determined by measuring at least one circulating cell biomarker expressed by the patient.

In a further embodiment, the circulating cell biomarker is selected from the group consisting of CD3+ cells, CD8+ T cells, CD4+ cells, CD56+NK cells, and CD14+ cells.

In a further embodiment, the amount of cabozantinib administered is sufficient to activate the immune system of a patient, increasing the number of circulating CD3+ cells and CD8+ T cells. In a further embodiment, the amount of cabozantinib administered is sufficient to activate the immune system of a patient, increasing the number of circulating CD3+ cells, CD8+ T cells, and CD4+ cells. In a further embodiment, the amount of cabozantinib administered is sufficient to activate the immune system of a patient, increasing the number of circulating CD3+ cells, CD8+ T cells, CD4+ cells, and CD56+NK cells. In another embodiment, the number of circulating CD3+ cells and CD8+ T cells is increased, and the number of CD+14 monocytes in the patient is decreased. In another embodiment, the number of circulating CD3+ cells, CD8+ T, and CD4+ cells is increased, and the number of CD+14 monocytes in the patient is decreased.

In another embodiment, the number of circulating CD3+ cells, CD8+ T, CD4+ cells, and CD56+NK cells is increased, and the number of CD+14 monocytes in the patient is decreased.

In another aspect, the invention relates to a method of treating triple negative breast cancer in a human patient, comprising administering to a patient in need of such treatment cabozantinib or a pharmaceutically acceptable salt thereof at a dose which activates circulating cell biomarkers, in combination with one or more additional therapies or agents. A number of therapies and agents are available or under development and are summarized, for instance, at www.cancerresearch.org/cancer-immunotherapy/impacting-all-cancers/breast-cancer (last visited Mar. 24, 2017).

In one embodiment, the additional therapy or agent is an immunotherapy or agent.

According to the Cancer Research Institute, although breast cancer has historically been considered immunologically silent, several preclinical and clinical studies suggest that immunotherapy has the potential to improve clinical outcomes for patients with breast cancer. See www.cancerresearch.org/cancer-immunotherapy/impacting-all-cancers/breast-cancer (last visited Mar. 24, 2017). Overall, immunotherapy holds several key advantages over conventional chemotherapeutic and targeted treatments directed at the tumor itself, that when combined with other therapies such as cabozantinib could be of significant TNBC patients. First, immunotherapy generally results in fewer side effects, enabling it to be administered for longer periods of time and/or in combination with other agents without added toxicity. Patients may also be less likely to develop resistance to immunotherapy because of the immune system's ability to target multiple cancer antigens simultaneously and adapt to changing cancer cells. Some immunotherapies that have shown promise in recent clinical trials are described below and are considered suitable for combination with cabozantinib.

Therapeutic Vaccines. Cancer vaccines are designed to elicit an immune response against tumor-specific or tumor-associated antigens, encouraging the immune system to attack cancer cells bearing these antigens. Several trials of vaccines, given alone or with other therapies, are currently enrolling breast cancer patients.

NeuVax (nelipepimut-S or E75) is under investigation to prevent breast cancer recurrence among patients with low-to-intermediate levels of HER2 expression (HER2 1+ and 2+) following surgery. A phase III trial (PRESENT) is now fully enrolled (NCT01479244). The trial has been granted a Special Protocol Assessment (SPA) by the FDA, meaning that, if the trial meets its pre-specified endpoint, it will fulfill the necessary criteria to file for regulatory approval. There is also a phase IIb trial of NeuVax for node-positive or triple-negative patients following standard-of-care treatment (NCT01570036), and a phase I/II among neoadjuvantly treated node-positive and -negative HER2 3+ patients not achieving a pathological complete response, or adjuvantly treated node-positive HER2 3+ patients (NCT02297698).

The following additional studies have been identified:

A phase I study of two vaccines—INO-1400, targeting TERT, which has been detected in more than 85% of all human cancers, and INO-9012, targeting interleukin 12 (IL-12), which enhances immune cell activity—for patients with select tumors, including breast cancer (NCT02327468).

A phase I trial of OBI-833 vaccine, which targets the Globo H marker that is commonly found on a variety of tumors cells, for patients with select metastatic cancers, including breast cancer (NCT02310464).

A phase I study of the MAG-Tn3 vaccine, which targets Tn carbohydrate antigen that is overexpressed in a number of tumor types, for patients with localized breast cancer at high-risk of relapse (NCT02364492).

A phase I trial of a HER2 peptide vaccine in patients with breast cancer (NCT02276300).

A phase I trial of a dendritic cell vaccine in patients with metastatic breast cancer (NCT02479230).

A phase I trial of a personalized vaccine in patients with persistent triple-negative breast cancer following neoadjuvant chemotherapy (NCT02348320).

A phase I trial of a personalized vaccine plus Poly-ICLC, a Toll-like receptor 3 agonist, in patients with persistent triple-negative breast cancer following neoadjuvant chemotherapy (NCT02427581).

Checkpoint Inhibitors/Immune Modulators. A promising avenue of clinical research in breast cancer is the use of immune checkpoint inhibitors. These treatments work by targeting molecules that serve as checks and balances in the regulation of immune responses. By blocking inhibitory molecules or, alternatively, activating stimulatory molecules, these treatments are designed to unleash or enhance pre-existing anti-cancer immune responses. Several checkpoint inhibitors, targeting multiple different checkpoints, are currently enrolling breast cancer patients:

### Pembrolizumab (Keytruda®, MK-3475): A PD-1 Antibody

A phase III trial for patients with metastatic triple-negative breast cancer, versus chemotherapy (NCT02555657).

A phase II trial for patients with breast cancer, with an HDAC inhibitor and anti-estrogen therapy (NCT02395627).

A phase II study for patients with triple-negative or hormone receptor-positive metastatic breast cancer, in combination with chemotherapy or anti-estrogen therapy (NCT02648477).

A phase II trial for patients with metastatic inflammatory breast cancer who have received prior chemotherapy with clinical response (NCT02411656).

A phase II trial for patients with metastatic triple-negative breast cancer (NCT02447003).

A phase I/II trial for patients with advanced cancer, including triple-negative breast cancer, combined with PLX3397, a tyrosine kinase inhibitor of KIT, CSF1R, and FLT3 (NCT02452424).

A phase I/II study for patients with advanced cancer, including breast cancer (NCT02318901).

A phase I/II trial for patients with advanced cancer, including breast cancer, in combination with chemotherapy (NCT02331251).

A phase I/II study in patients with triple-negative breast cancer, combined with niraparib, a PARP inhibitor (NCT02657889).

A phase I/II trial for patients with metastatic triple-negative breast cancer, in combination with chemotherapy (NCT02513472).

A phase I study in patients with refractory cancer, including triple-negative breast cancer, combined with MGA217, an antibody that targets B7-H3 (NCT02475213).

A phase I study for patients with advanced tumors, including triple-negative breast cancer, in combination with a JAK inhibitor, INCB039110, or a PI3K-delta inhibitor, INCB050465 (NCT02646748).

A phase I neoadjuvant trial for patients with triple-negative breast cancer, in combination with chemotherapy (NCT02622074).

A phase I study for patients with breast cancer that has metastasized to the bones (NCT02303366).

Nivolumab (Opdivo®): A PD-1 Antibody +/− Ipilimumab (Yervoy®), A CTLA-4 Antibody:

A phase II study of nivolumab after induction treatment for patients with triple-negative breast cancer (NCT02499367).

A phase I trial to test nivolumab and ipilimumab, plus entinostat, an HDAC inhibitor, for patients with locally advanced or metastatic HER2-negative breast cancer (NCT02453620).

A phase I study to test ipilimumab (Yervoy) combined with MGA217, an antibody that targets B7-H3, in patients with refractory cancer, including triple-negative breast cancer (NCT02381314).

A phase I study of nivolumab in combination with chemotherapy for patients with recurrent metastatic breast cancer (NCT02309177).

Durvalumab (MEDI4736), A PD-L1 Antibody +/− Tremelimumab: A CTLA-4 Antibody:

A phase II trial of durvalumab, tremelimumab, or the combination for patients with advanced tumors, including triple-negative breast cancer (NCT02527434).

A phase II study of durvalumab and tremelimumab in patients with metastatic HER2-negative breast cancer (NCT02536794).

A phase I/II trial of durvalumab, tremelimumab, and Poly-ICLC, a Toll-like receptor 3 agonist, in patients with advanced, measurable cancers, including locally recurrent breast cancer (NCT02643303). This is sponsored by the Cancer Research Institute.

A phase I/II trial of neoadjuvant durvalumab with chemotherapy for stage 1-3 triple-negative breast cancer (NCT02489448).

A phase I/II trial of durvalumab in combination with olaparib, a PARP inhibitor, or cediranib, a VEGF inhibitor, in patients with advanced solid tumors, including breast cancer (NCT02484404).

A phase I/II trial of durvalumab plus epacadostat (INCB024360), an IDO inhibitor, in patients with select advanced tumors, including triple-negative breast cancer (NCT02318277). IDO is expressed by a number of tumor types and correlates with poor prognosis.

A phase I/II study of durvalumab plus ibrutinib, a BTK inhibitor, in patients with relapsed or refractory tumors, including breast cancer (NCT02403271).

A phase I trial of durvalumab for patients with breast cancer, in combination with selumetinib, an inhibitor of MEK 1 and 2 (NCT02586987).

A phase I study of durvalumab plus tremelimumab for patients with breast cancer (NCT02639026).

A phase I study of durvalumab and tremelimumab for patients with advanced solid tumors, including non-triple-negative breast cancer (NCT01975831). This is sponsored by the Cancer Research Institute.

Tremelimumab

A pilot study of tremelimumab and brain irradiation for patients with breast cancer that has metastasized to the brain (NCT02563925).

Atezolizumab (MPDL3280A): A PD-L1 Antibody:

A phase III trial for patients with previously untreated metastatic triple-negative breast cancer, in combination with chemotherapy (NCT02425891).

A phase II first-line neoadjuvant trial for patients with triple-negative breast cancer, along with chemotherapy (NCT02530489).

A phase I/II study in patients with advanced cancer, including triple-negative breast cancer, in combination with varlilumab (CDX-1127), an anti-CD27 antibody (NCT02543645).

A phase I trial for patients with HER2-positive breast cancer, given with HER2 inhibitors (NCT02605915).

A phase I trial for patients with select advanced cancers, including breast cancer (NCT01375842).

A phase I study of CPI-444, which targets the adenosine-A2A receptor that suppresses the anti-tumor activity of immune cells, +/− atezolizumab for patients with advanced cancer, including triple-negative breast cancer (NCT02655822).

Other Drugs:

A phase II study of IMP321, a LAG-3 fusion protein, in patients with hormone receptor-positive metastatic breast cancer, in combination with chemotherapy (NCT02614833).

A phase I/II trial of MEDI6469, an anti-OX40 antibody, for patients with stage 4 breast cancer who have failed prior hormone or chemotherapy (NCT01642290). OX40 is a costimulatory molecule expressed after T cell activation that enhances T cell survival and anti-cancer effector function.

A phase I/II trial of PDR001, a PD-1 antibody, in patients with advanced cancers, including triple-negative breast cancer (NCT02404441).

A phase I study to test MGD009, a B7-H3×CD3 DART protein, in patients with unresectable or metastatic B7-H3-expressing cancer, including breast cancer (NCT02628535).

Adoptive Cell Therapy:

Another avenue of immunotherapy for breast cancer is adoptive T cell transfer. In this approach, T cells are removed from a patient, genetically modified or treated with chemicals to enhance their activity, and then re-introduced into the patient with the goal of improving the immune system's anti-cancer response. Several trials of adoptive T cell transfer techniques are currently under way for patients with breast cancer, including:

A phase I trial of chimeric antigen receptor (CAR) T cells targeting cMet—which is abnormally activated in cancer and correlates with poor prognosis—is being tested in metastatic breast cancer refractory to at least one standard therapy or newly diagnosed patients with operable triple negative breast cancer (NCT01837602).

A phase I study of immune cells engineered to target the mesothelin protein, which is overexpressed in certain cancers, in patients with advanced cancer, including breast cancer (NCT02414269).

A phase I study of T cells engineered to recognize the NY-ESO-1, MAGE-A4, PRAME, survivin, and SSX markers in patients with solid tumors, including breast cancer (NCT02239861).

Oncolytic Virus Therapies:

Oncolytic virus therapy uses a modified virus that can cause tumor cells to self-destruct and generate a greater immune response against the cancer.

A phase I/II trial of PexaVec (JX-594), a virus engineered to secrete GM-CSF and delete a kinase gene that is typically seen on cancer cells with a mutated RAS or p53 pathway, for patients with advanced breast cancer (NCT02630368).

Antibodies:

Monoclonal antibodies are molecules, generated in the lab, that target specific antigens on tumors. Many antibodies are currently used in cancer treatment, and some appear to generate an immune response.

A phase III study of margetuximab (MGAH22), an anti-HER2 antibody, plus chemotherapy versus trastuzumab (Herceptin®) plus chemotherapy in patients with HER2-positive metastatic breast cancer (NCT02492711).

A phase II study of margetuximab (MGAH22) in patients with relapsed or refractory advanced breast cancer whose tumors express HER2 at the 2+ level and lack HER2 gene amplification by FISH (NCT01828021).

A phase II trial of glembatumumab vedotin (CDX-011), an antibody-drug conjugate, in patients with advanced triple-negative breast cancer whose cancer cells make a protein called glycoprotein NMB, to which CDX-011 binds (NCT01997333).

A phase I/II trial of TRC105, an antibody targeting endoglin, which is a protein that is overexpressed on endothelial cells and is essential for angiogenesis, the process of new blood vessel formation, in patients with hormone receptor-positive and HER2-negative breast cancer (NCT02520063).

A phase II trial of MCS110, an antibody that targets the macrophage colony-stimulating factor, in patients with advanced triple-negative breast cancer (NCT02435680).

A pilot study of QBX258, which targets interleukin 4 (IL-4) and interleukin 13 (IL-13), in patients with stage 1-2 breast cancer related lymphedema (NCT02494206).

Adjuvant Immunotherapies:

Adjuvants are substances that are either used alone or combined with other immunotherapies to boost the immune response. Some adjuvant immunotherapies use ligands—molecules that bind to proteins such as receptors—to help control the immune response. These ligands can be either stimulating (agonists) or blocking (antagonists).

A phase I/II trial of durvalumab plus epacadostat (INCB024360), an IDO inhibitor, in patients with select advanced tumors, including triple-negative breast cancer (NCT02318277). IDO is expressed by a number of tumor types and correlates with poor prognosis.

A phase I trial of motolimod (VTX-2337), a Toll-like receptor 8 (TLR8) agonist, in patients with metastatic, persistent, recurrent, or progressive solid tumors, including breast cancer (NCT02650635).

A phase I study of entinostat (KHK2375), a small molecule drug that targets both cancer cells and immune regulatory cells, in patients with advanced or recurrent breast cancer (NCT02623751).

Cytokines:

Cytokines are messenger molecules that help control the growth and activity of immune system cells.

A phase I/II study of interleukin 12 (IL-12) in patients with metastatic breast cancer (NCT02423902).

In another aspect, the invention relates to a method of treating HER2 triple negative breast cancer in a human patient, comprising administering to a patient in need of such treatment cabozantinib or a pharmaceutically acceptable salt thereof at a dose which activates circulating cell biomarkers, in combination with one or more additional agents.

In one embodiment of this aspect, the one or more circulating biomarkers is selected from the group consisting of CD31 cells, CD31 CD4−CD81 T lymphocytes, CD141 monocytes, CD3+CD4+CD8-T lymphocytes, CD3−CD561 NK lymphocytes, CD1331 progenitor/stem cells, CD4+CD25+ regulatory T cells, CD4+CD127+ memory T cells, and CD3+CD56+ NKT cells.

In another embodiment, the HER2 triple negative breast cancer is HER3+ or FISH-positive breast cancer.

In another embodiment, the one or more additional agents is an immune modulator selected from the group consisting of trastuzumab, pertuzumab, ado-trastuzumab emantine, lapatinib, fulvestrant, pemborlizumab, nivolumab, ipilimumab, durvalumab, tremelimumab, epacadostat, atezolizumab, and PDR001, as described above.

In another aspect, the invention relates to a method of treating triple negative breast cancer in a human patient, comprising administering to a patient in need of such treatment cabozantinib or a pharmaceutically acceptable salt thereof at a dose which activates circulating cell biomarkers, in combination with one or more additional therapies or agents.

In one embodiment of this aspect, the one or more circulating biomarkers is selected from the group consisting of CD31 cells, CD31 CD4-CD81 T lymphocytes, CD141 monocytes, CD3+CD4+CD8-T lymphocytes, CD3-CD561 NK lymphocytes, CD1331 progenitor/stem cells, CD4+CD25+ regulatory T cells, CD4+CD127+ memory T cells, and CD3+CD56+ NKT cells.

In another embodiment, the one or more additional agents is selected from the group consisting of trastuzumab, pertuzumab, ado-trastuzumab emantine, and lapatinib, as described above.

In another embodiment, the one or more additional agents is a vaccine, wherein the vaccine is selected from the group consisting of nelipepimut-S, INO-1400, INO-9012, OBI-833, MAG-Tn3 HER-2 peptide vaccine, a personalized vaccine, and POLY-ICLC, as described above.

In another embodiment, the one or more additional agents is selected from the group consisting of the LAG fusion protein IMP321, the anti-0X40 antibody MEDI6469, and the B7-H3×CD3 DART protein MGD009, as described above.

In another embodiment, the one or more additional therapy is selected from adoptive T-cell transfer, oncolyitic virus therapy, antibodies, adjuvant immunotherapies, and cytokines, as described above.

The invention will now be illustrated by following non-limiting embodiments.

Embodiment 1. A method of treating triple negative breast cancer in a human patient, comprising administering to the patient an amount of cabozantinib or a pharmaceutically acceptable salt thereof, wherein the amount of cabozantinib is sufficient to activate one or more circulating biomarkers of the immune system.

Embodiment 2. The method of embodiment 1, wherein the one or more circulating biomarkers is selected from the group consisting of CD31 cells, CD31 CD4-CD81 T lymphocytes, CD141 monocytes, CD3+CD4+CD8-T lymphocytes, CD3-CD561 NK lymphocytes, CD1331 progenitor/stem cells, CD4+CD25+ regulatory T cells, CD4+CD127+ memory T cells, and CD3+CD56+ NKT cells.

Embodiment 3. The method of embodiments 1-2, wherein cabozantinib is administered as cabozantinib (S)-malate.

Embodiment 4. The method of embodiments 1-3, wherein the cabozantinib (S)-malate is administered as a tablet formulation comprising approximately (% w/w):

30-32 percent by weight of cabozantinib, (S)-malate salt;

38-40 percent by weight of microcrystalline cellulose;

18-22 percent by weight of lactose;

2-4 percent by weight of hydroxypropyl cellulose;

4-8 percent by weight of croscarmellose sodium;

0.2-0.6 percent by weight of colloidal silicon dioxide;

0.5-1 percent by weight of magnesium stearate; and further comprising:

a film coating material comprising hypromellose, titanium dioxide, triacetin, and iron oxide yellow.

Embodiment 5. The method of embodiments 1-4, wherein the cabozantinib (S)-malate is administered as a tablet formulation comprising approximately (% w/w):

31-32 percent by weight of cabozantinib, (S)-malate salt;

39-40 percent by weight of microcrystalline cellulose;

19-20 percent by weight of lactose;

2.5-3.5 percent by weight of hydroxypropyl cellulose;

5.5-6.5 percent by weight of croscarmellose sodium;

0.25-0.35 percent by weight of colloidal silicon dioxide;

0.7-0.8 percent by weight of magnesium stearate; and further comprising:

3.9-4.1 percent by weight of a film coating material comprising hypromellose, titanium dioxide, triacetin, and iron oxide yellow.

Embodiment 6. The method of embodiments 1-5, wherein cabozantinib (S)-malate is administered as a tablet formulation containing 20, 40, or 60 mg of cabozantinib.

Embodiment 7. The method of embodiments 1-6, wherein cabozantinib (S)-malate is administered as a tablet formulation selected from the group consisting of:

| | Theoretical Quantity (mg/unit dose) | | |
|---|---|---|---|
| Ingredient | 20-mg Tablet* | 40-mg Tablet* | 60-mg Tablet* |
| Cabozantinib (S)-malate | 25.34 | 50.69 | 76.03 |
| Microcrystalline Cellulose, PH-102 | 31.08 | 62.16 | 93.24 |
| Lactose Anhydrous, 60M | 15.54 | 31.07 | 46.61 |
| Hydroxypropyl Cellulose, EXF | 2.400 | 4.800 | 7.200 |
| Croscarmellose Sodium | 4.800 | 9.600 | 14.40 |
| Colloidal Silicon Dioxide | 0.2400 | 0.4800 | 0.7200 |
| Magnesium Stearate (Non-Bovine) | 0.6000 | 1.200 | 1.800 |
| Opadry ® Yellow (03K92254) | 3.200 | 6.400 | 9.600 |
| Total tablet weight | 83.20 | 166.4 | 249.6 |

*Free Base Equivalent (FBE)

Embodiment 8. The method of embodiments 1-7, wherein the cabozantinib (S)-malate is administered once daily.

Embodiment 9. The method of embodiments 1-8, wherein the amount of cabozantinib that is administered once daily is 60 mg.

Embodiment 10. A method of treating triple negative breast cancer in a human patient, comprising administering to a patient in need of such treatment cabozantinib or a pharmaceutically acceptable salt thereof at a dose which activates circulating cell biomarkers.

Embodiment 11. The method of embodiment 10, wherein circulating cell biomarker activation is determined by measuring at least one circulating cell biomarker expressed by the patient.

Embodiment 12. The method of embodiments 10-11, wherein the circulating cell biomarker is selected from the group consisting of CD3+ cells, CD8+ T cells, CD4+ cells, CD56+NK cells, and CD14+ cells.

Embodiment 13. The method of embodiments 10-12, wherein cabozantinib is administered as cabozantinib (S)-malate.

Embodiment 14. The method of embodiments 10-13, wherein the cabozantinib (S)-malate is administered as a tablet formulation comprising approximately (% w/w):

30-32 percent by weight of cabozantinib, (S)-malate salt;

38-40 percent by weight of microcrystalline cellulose;

18-22 percent by weight of lactose;

2-4 percent by weight of hydroxypropyl cellulose;

4-8 percent by weight of croscarmellose sodium;

0.2-0.6 percent by weight of colloidal silicon dioxide;

0.5-1 percent by weight of magnesium stearate; and further comprising:

a film coating material comprising hypromellose, titanium dioxide, triacetin, and iron oxide yellow.

15. The method of embodiments 10-14, wherein the cabozantinib (S)-malate is administered as a tablet formulation comprising approximately (% w/w):

31-32 percent by weight of cabozantinib, (S)-malate salt;

39-40 percent by weight of microcrystalline cellulose;

19-20 percent by weight of lactose;

2.5-3.5 percent by weight of hydroxypropyl cellulose;

5.5-6.5 percent by weight of croscarmellose sodium;

0.25-0.35 percent by weight of colloidal silicon dioxide;

0.7-0.8 percent by weight of magnesium stearate; and further comprising:

3.9-4.1 percent by weight of a film coating material comprising hypromellose, titanium dioxide, triacetin, and iron oxide yellow.

Embodiment 16. The method of embodiments 10-15, wherein cabozantinib (S)-malate is administered as a tablet formulation containing 20, 40, or 60 mg of cabozantinib.

Embodiment 17. The method of embodiments 10-16, wherein cabozantinib (S)-malate is administered as a tablet formulation selected from the group consisting of:

| | Theoretical Quantity (mg/unit dose) | | |
|---|---|---|---|
| Ingredient | 20-mg Tablet* | 40-mg Tablet* | 60-mg Tablet* |
| Cabozantinib (S)-malate | 25.34 | 50.69 | 76.03 |
| Microcrystalline Cellulose, PH-102 | 31.08 | 62.16 | 93.24 |
| Lactose Anhydrous, 60M | 15.54 | 31.07 | 46.61 |
| Hydroxypropyl Cellulose, EXF | 2.400 | 4.800 | 7.200 |
| Croscarmellose Sodium | 4.800 | 9.600 | 14.40 |
| Colloidal Silicon Dioxide | 0.2400 | 0.4800 | 0.7200 |
| Magnesium Stearate (Non-Bovine) | 0.6000 | 1.200 | 1.800 |
| Opadry ® Yellow (03K92254) | 3.200 | 6.400 | 9.600 |
| Total tablet weight | 83.20 | 166.4 | 249.6 |

*Free Base Equivalent (FBE)

Embodiment 18. The method of embodiments 10-17, wherein the cabozantinib (S)-malate is administered once daily.

Embodiment 19. The method of embodiments 10-18, wherein the amount of cabozantinib that is administered once daily is 60 mg.

Embodiment 20. A method of treating HER2 triple negative breast cancer in a human patient, comprising administering to a patient in need of such treatment cabozantinib or a pharmaceutically acceptable salt thereof at a dose which activates circulating cell biomarkers, in combination with one or more additional agents.

Embodiment 21. The method of embodiment 20 wherein the one or more circulating biomarkers is selected from the group consisting of CD31 cells, CD31 CD4-CD81 T lymphocytes, CD141 monocytes, CD3+CD4+CD8-T lymphocytes, CD3-CD561 NK lymphocytes, CD1331 progenitor/stem cells, CD4+CD25+ regulatory T cells, CD4+CD127+ memory T cells, and CD3+CD56+ NKT cells.

Embodiment 22. The method of embodiment 20, wherein the HER2 triple negative breast cancer is HER3+ or FISH-positive breast cancer.

Embodiment 23. The method of embodiment 20, wherein the one or more additional agents is an immune modulator selected from the group consisting of trastuzumab, pertuzumab, ado-trastuzumab emantine, lapatinib, fulvestrant, pemborlizumab, nivolumab, ipilimumab, durvalumab, tremelimumab, epacadostat, atezolizumab, and PDR001.

Embodiment 24. A method of treating triple negative breast cancer in a human patient, comprising administering to a patient in need of such treatment cabozantinib or a pharmaceutically acceptable salt thereof at a dose which activates circulating cell biomarkers, in combination with one or more additional therapies or agents.

Embodiment 25. The method of embodiment 24, wherein the one or more circulating biomarkers is selected from the group consisting of CD31 cells, CD31 CD4-CD81 T lymphocytes, CD141 monocytes, CD3+CD4+CD8-T lymphocytes, CD3-CD561 NK lymphocytes, CD1331 progenitor/stem cells, CD4+CD25+ regulatory T cells, CD4+CD127+ memory T cells, and CD3+CD56+ NKT cells.

Embodiment 26. The method of embodiment 24, wherein the one or more additional agents is selected from the group consisting of trastuzumab, pertuzumab, ado-trastuzumab emantine, and lapatinib.

Embodiment 27. The method of embodiment 24 wherein the one or more additional agents is a vaccine, wherein the vaccine is selected from the group consisting of nelipepimut-S, INO-1400, INO-9012, OBI-833, MAG-Tn3 HER-2 peptide vaccine, a personalized vaccine, and POLY-ICLC.

Embodiment 28. The method of embodiment 24, wherein the one or more additional agents is selected from the group consisting of the LAG fusion protein IMP321, the anti-0X40 antibody MEDI6469, and the B7-H3×CD3 DART protein MGD009.

Embodiment 29. The method of embodiment 24, wherein the one or more additional therapy is selected from the group consisting of adoptive T-cell transfer, oncolytic virus therapy, antibodies, adjuvant immunotherapies, and cytokines.

Embodiment 30. A method of treating triple negative breast cancer in a human patient having a baseline plasma concentration of sMET that is greater than the median baseline plasma concentration of sMET in humans, comprising administering to the patient an amount of cabozantinib or a pharmaceutically acceptable salt thereof, wherein the amount of cabozantinib is sufficient to activate the immune system.

Embodiment 31. The method of embodiment 30, wherein the baseline plasma concentration of sMET greater than or equal to 795 mg/mL median value.

Embodiment 32. The method of embodiment 31, wherein progression free survival of patients having a baseline plasma concentration of sMET of greater than or equal to 795 mg/mL median value is extended as compared to patients having a baseline plasma concentration of sMET of less than 795 mg/mL median value.

The invention will now be illustrated by the following non-limiting examples.

### EXAMPLES

### Cabozantinib Treatment Induces Significant Changes in Circulating Immune Cell Populations in Patients with Metastatic Triple-Negative Breast Cancer (TNBC)

Purpose: To evaluate the changes in circulating immune cell populations in patients enrolled in a phase II study of cabozantinib (XL184), an inhibitor of multiple receptor tyrosine kinases, including MET and VEGFR2, for metastatic TNBC. (NCT02260531)

Experimental design: In this single-arm, two-stage phase 2 study, patients with metastatic TNBC with measurable disease by RECIST and up to 3 lines of prior chemotherapy for metastatic disease received cabozantinib 60 mg daily on a 21-day cycle. Patients were restaged 6 weeks following treatment initiation and every 9 weeks thereafter. The primary endpoint was objective response rate (ORR). Predefined secondary endpoints included progression free survival (PFS) and toxicity. Here, we examined cellular biomarkers using flow cytometry in serial blood samples collected at days 0 (baseline/pre-treatment), 8, 22, 43, and 64 of cabozantinib treatment. Mixed effect models were used to evaluate the changes of biomarker levels over time from baseline to day 64. Wilcoxon signed rank test were used to evaluate whether the change of biomarker levels from baseline to day 8 were different by clinical benefit. Adjusted p-values controlling false discovery rate were used to adjust for multiple comparisons.

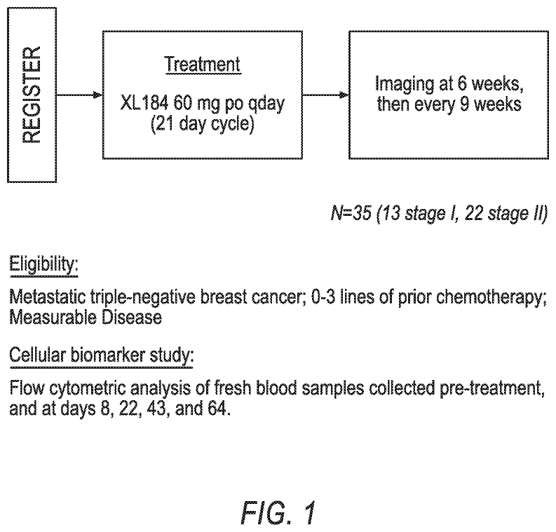

The experimental design is depicted in FIG. 1.

Results: The analysis included all 35 patients who initiated protocol therapy. As previously reported (ASCO 2015), the ORR was 11%, the clinical benefit rate (PR+SD) at 15 weeks was 34% (95% CI 19-52%) and the median PFS was 2.0 months (95%, CI 1.3-3.3). From baseline to day 64, there were significant increases in the number of circulating CD3+ cells and CD8+ T cells, and decreases in CD14+ monocytes (all p<0.05) at all time-points. There was a trend for increase in CD4+ cells (p=0.08) and CD56+ NK cells (p=0.07) but no significant changes in the fraction of CD133+ progenitor/stem cells, CD4+CD25+ Tregs, CD4+CD127+ memory T cells and CD3+CD56+ NKT cells. The changes of biomarker levels from baseline to day 8 were not significantly different between patients with and without clinical benefit.

Summary: Analysis of circulating cell biomarkers showed that cabozantinib induces systemic changes consistent with activation of the immune system in metastatic TNBC patients. These hypothesis-generating data support further studies of cabozantinib with immunotherapies in this patient population.

Experimental Details

Patients: Patient characteristics are summarized in Table 1. Patients 18 years of age or older with measurable metastatic TNBC were eligible. Triple-negative status was defined as estrogen receptor-negative (ER-) (<10% staining by immunohistochemistry [IHC]), progesterone receptor-negative (PR-) (<10% staining by IHC), and HER2-negative (0 or 11 by IHC or fluorescence in situ hybridization [FISH]<2.0). Patients had measurable disease by Response Evaluation Criteria In Solid Tumors (RECIST) version 1.1 and may have received 0 to 3 prior chemotherapeutic regimens for mTNBC. They were required to be off any myelosuppressive agent for 21 days before initiation of cabozantinib and must have discontinued all biologic therapy and radiation therapy at least 14 days before initiation of study treatment. Patients were required to have an Eastern Cooperative Oncology Group (ECOG) performance status #2 and were required to have availability of formalin-fixed, paraffin-embedded (FFPE) tumor tissue. Key exclusion criteria included the following: receipt of another investigational agent within 14 days of the first dose of the study drug; prior receipt of a MET inhibitor other than tivantinib (ARQ-197); known brain metastases that were untreated, symptomatic, or required therapy to control symptoms; and corrected QT.470 milliseconds. Research was approved by local human research protections programs and institutional review boards, and studies were conducted in accordance with the Declaration of Helsinki. Patients were restaged 6 weeks following treatment initiation and every 9 weeks thereafter.

TABLE 1

| Characteristics | Value |
|---|---|
| Median age, yr (range) | 50 (31-78) |
| Female sex, [symbol] (%) | 35 (100) |
| Race, [symbol] (%) | |
| White | 32 (92) |
| African American | 3 (9) |
| Triple negative primary tumor, n (%) | 25 (72) |
| Triple negative metastatic tumor, [symbol] (%) | 33 (94) |
| Prior line of chemotherapy for resectable disease | |
| preceding metastases [symbol] , n (%) | |
| 0 | 19 (54) |
| 1 | 6 (17) |
| 2 | 1 (3) |
| Not applicable | 9 (25) |
| Prior lines of chemotherapy for metastatic or | |
| unresectable disease [symbol] , [symbol] (%) | |
| 0 | 6 (17) |
| 1 | 18 (51) |
| 2 | 4 (11) |
| 3 | 7 (20) |
| ECOG performance status | |
| 0 | 26 (74) |
| 1 | 8 (23) |
| 2 | 1 (3) |
| Median metastatic sites (range) | 3 (1-6) |
| Sites of metastatic disease, [symbol] (%) | |
| Lung | 18 (51) |
| Pleural effusion | 2 (6) |
| Liver | 12 (34) |
| Bone | 13 (37) |
| Breast or chest wall | 16 (46) |
| Lymph nodes | 26 (74) |
| Others | 15 (43) |

[symbol]

—35. [symbol]
including chemotherapy [symbol] [symbol]
Including chemotherapy for local [symbol] completely removed by surgery.
Abbreviations: ECOG, [symbol] Cooperative Oncology Group. [symbol]
indicates data missing or illegible when filed

Study Design and Treatment: As indicated, this was a single-arm, two-stage phase II study assessing the efficacy of cabozantinib monotherapy in patients with mTNBC. Treatment consisted of oral dosing of cabozantinib at 60 mg daily over a 21-day cycle. Patients underwent radiographic restaging at 6 weeks and every 9 weeks thereafter. Patients with complete or partial RECIST responses continued to receive study treatment, whereas those with progressive disease were taken off study. Dose reductions for toxicity occurred if patients experienced grade 3 or 4 neutropenia or thrombocytopenia, or nonhematologic adverse events. From the starting dose of 60 mg daily, doses were reduced as needed to 40 and 20 mg daily. For the purposes of determining the effect of cabozantinib treatment on pain and analgesic medication use, pain was assessed by a participant-reported questionnaire, and daily analgesic medication usage was recorded. These were completed at baseline and during week 3, 6, and every 6 weeks thereafter until the date of the participant's last follow-up visit.

The primary endpoint was the activity of cabozantinib, as defined by objective response rate(ORR)in patients with mTNBC. Predefined secondary endpoints included progression-free survival (PFS), toxicity, and pain. Correlative studies included analysis of MET and phospho-MET expression in archival tumor tissue, and molecular and cellular biomarkers of cabozantinib. Cellular biomarkers were examined using flow cytometry in serial blood samples collected at days 0 (baseline/pre-treatment), 8, 22, 43, and 64 of cabozantinib treatment. Mixed effect models were used to evaluate the changes of biomarker levels over time from baseline to day 64. Wilcoxon signed rank test were used to evaluate whether the change of biomarker levels from baseline to day 8 were different by clinical benefit. Adjusted p-values controlling false discovery rate were used to adjust for multiple comparisons.

Fluorescence In Situ Hybridization (FISH) Assessment of MET Amplification in Tissue: A MET FISH probe labeled with SpectrumRed and a CEP7 reference probe labeled with Spectrum Green were purchased from Abbott Molecular (Des Plaines, Ill., www.abbott molecular.com). FISH was performed following standard protocols. Briefly, 5 micrometer tissue slides were baked overnight at 60° C., deparaffinized, treated in 1% sodium borohydride for 4 hours, and heated in pressure cooker for 20 minutes in citrate buffer (pH 6). After treatment with 150 microgram/mL solution of proteinase K, slides were fixed in 1% neutral-buffered formalin, and denatured in 70% formamide for 4 minutes at 72° C. Probes were denatured for 5 minutes at 80° C. and incubated for 30 minutes at 37° C. for preannealing. Hybridization was carried out overnight at 37° C. Posthybridization slide washes were carried out for 20 minutes in 50% formamide/2×standard saline citrate (SSC) at 45° C., followed by 5 minutes wash in 1×SSC at 45° C. FISH signal evaluation and acquisition were performed manually by using filter sets and software developed by Applied Spectral Imaging (Carlsbad, Calif., www.spectral-imaging.com). Several fields with at least 50 tumor cells total were captured, and ratio of MET to CEP7 signal numbers was calculated. An assessment of ploidy was made by visual screening of all tumor area, and cells with the maximum number of signals were recorded. MET amplification was defined as a MET/CEP7 ratio of ≥2. Samples with a MET/CEP7 ratio between 1.5 and 2 were defined as having relative MET gain. Samples with a MET/CEP7 ratio of 1, but with more than two copies of each probe, were deemed to have polysomy of chromosome 7.

Assessment of MET Amplification in Circulating Tumor Cells: Circulating tumor cells (CTCs) were enriched from 7.5 mL of a patient's whole blood at the Circulating Tumor Cell Core Facility (Brigham and Women's Hospital, Boston, Mass., www.brighamandwomens.org) by using the Circulating Tumor Cell Profile Kit (Veridex/Janssen Diagnostics, Raritan, N.J., www.janssen.com). Processed samples were received as cells suspended in 900 mL of buffer. Equal volume of PBS was added before tubes were spun down at 200 g for 8 minutes. Supernatant was carefully removed, leaving approximately 60 mL of buffer. Cell pellets were gently resuspended, and the suspension was applied on the labeled slide and allowed to dry in the vacuum dessicator at room temperature. Slides were placed in methanol at 220° C. for aging and storage.

For FISH, dried slides were treated in 23 SSC at 37° C. for 30 minutes, followed by 10 minutes of treatment with 0.002% pepsin solution in 0.01MHCl at 37° C. and 15 minutes of fixation in 1% formalin at room temperature. Slides were dehydrated in the series of ethanols, dried, and codenatured with MET/CEP7 FISH probe (Kreatech/Leica Microsystems Inc., Buffalo Grove, Ill., www.leica-microsystems.com) on an 80° C. plate for 2 minutes. Hybridization was carried out at 37° C. overnight, followed by a 0.43 SSC/0.3% Igepal wash at 72° C. for 3 minutes and a 23 SSC/0.1% Igepal wash at room temperature for 1 minute. Slides were dehydrated in the series of ethanols and dried before application of Vectashield mounting medium with 49,6-diamidino-2-phenylindole (Vector Laboratories Inc., Burlingame, Calif., vectorlabs.com). FISH signal evaluation and acquisition were performed manually by using filter sets and software developed by Applied Spectral Imaging.

Circulating Biomarker Assays: Potential biomarkers of cabozantinib activity were identified by measuring plasma proteins at baseline, on day 8 of therapy, on day 1 of each cycle of therapy, and, if available, at the time of progression. Eight milliliters of blood was collected in purple top (plasma EDTA) vacutainers and shipped on wet ice to a Clinical Laboratory Improvement Amendments-certified core in the Steele Laboratories (Massachusetts General Hospital), where whole blood was separated by centrifugation into cellular fraction and plasma. The fraction of stem/progenitor cell, lymphocyte, and myeloid populations of total circulating mononuclear cells were counted by flow cytometry using a LSR-II cytometer and FACSDiva software in fresh blood samples using the following markers: CD3, CD4, CD8, CD14, CD25, CD34, CD45, CD56, CD127, and CD133 (Becton Dickinson, Franklin Lakes, N.J., www.bd.com). Plasma was prepared in the standard fashion and stored at −78° C. until collection and analysis of all samples. The biomarkers measured included VEGF, placental growth factor (P1GF), VEGF-C, VEGF-D, soluble VEGFR1 (sVEGFR1), basic fibroblast growth factor (bFGF), and sTie-2 (using a 7-plex Growth Factor array) and granulocyte-macrophage colony stimulating factor (GM-CSF), interferon gamma (IFN-g),tumor necrosis factor alpha (TNF-a), and interleukin-lbeta (IL-1b), IL-2, IL-6, IL-8, IL-10, and IL-12 heterodimer p70 (using a 9-plex Inflammatory Factor array; both Meso Scale Discovery, Gaithersburg, Md., www.mesoscale.com); and HGF, sMET, carbonic anhydrase IX (CAIX), stromal cell-derived factor 1 a (SDF1a), and sVEGFR2 by single analyte enzyme-linked immunosorbent assay (R&D Systems, Minneapolis, Minn., www.rndsystems.com).

Statistical Analysis: This study used Simon optimal two-stage design to control type I error at 10% and have at least 90% power to detect the acceptable response rate. By study design, 13 participants were to be enrolled in the first stage. If there was at least 1 response, accrual was to continue to the second stage, where an additional 22 patients were to be enrolled. If there were at least 4 responses among the 35 total patients, the regimen was to be considered worthy of further study. With a true response rate of 5%, the chance that the regimen would be declared worthy of further study was 10%, and with a true response rate of 20%, the chance that the regimen would be declared worthy of further study was 90%.

Objective response was evaluated by using RECIST1.1. Per protocol, patients who do not achieve a confirmed complete response (CR) or confirmed partial response (PR) were considered non-responders. Objective response rate was reported with 95% confidence interval (CI) for the two stage designs. PFS and 95% CI were described using Kaplan-Meier methods. PFS was defined as the duration of time from study entry to time of objective disease progression, or time of death from any cause, whichever came first. For patients who were taken off of protocol treatment for any reason other than progression, the date of PFS was censored at the date of last staging study (either on or off protocol therapy) on which the patient was documented not to have progressed, or the date of initiation of alternative anticancer therapy, whichever came first. Clinical benefit rate was included as an exploratory analysis. Clinical benefit included confirmed CR, PR, and stable disease (SD) of 15 weeks or longer. If patients had unconfirmed PR followed by SD, they were considered to receive clinical benefit.

Descriptive statistics were used to summarize biomarker values at protocol-specific time points. The Wilcoxon ranked sum test evaluated the difference of baseline biomarker values between patients who did or did not experience clinical benefit. The Wilcoxon signed rank test assessed biomarker change from day 1 to 8. Mixed effects linear models assessed the change in biomarker values at days 1, 8, 22, 43, and 64; values beyond day 64 were not analyzed because of the small number of patients still on protocol. In the mixed effects linear model, the fixed effects were times of assessment, and patients were entered as a random effect. Logarithmic transformation was used to achieve normality, when applicable. Baseline biomarkers were stratified by using the median values for the entire cohort. The log-rank test compared PFS among patients with low or high baseline sMET. All tests were conducted with two-sided a5 0.05. The Benjamini-Hochberg procedure was used to adjust p values to control the false discovery rate from evaluating multiple circulating biomarkers.

Analysis of Results

Patients: The analysis included all 35 patients who initiated protocol therapy. Median age was 50 years (range 31-78); patients had received 0 (n=5 6; 17%), 1 (n=5 18; 51%), 2 (n=5 4; 11%), or 3 (n=5 7; 20%) lines of chemotherapy for mTNBC (Table 1). The median number of metastatic sites was 3 (range 1-6). The most common sites of metastatic disease were regional lymph nodes (n=5 26; 74%), lung (n=5 18; 51%), breast or chest wall (n=5 16; 46%), bone (n=5 13; 37%), and liver (n=5 12; 34%).

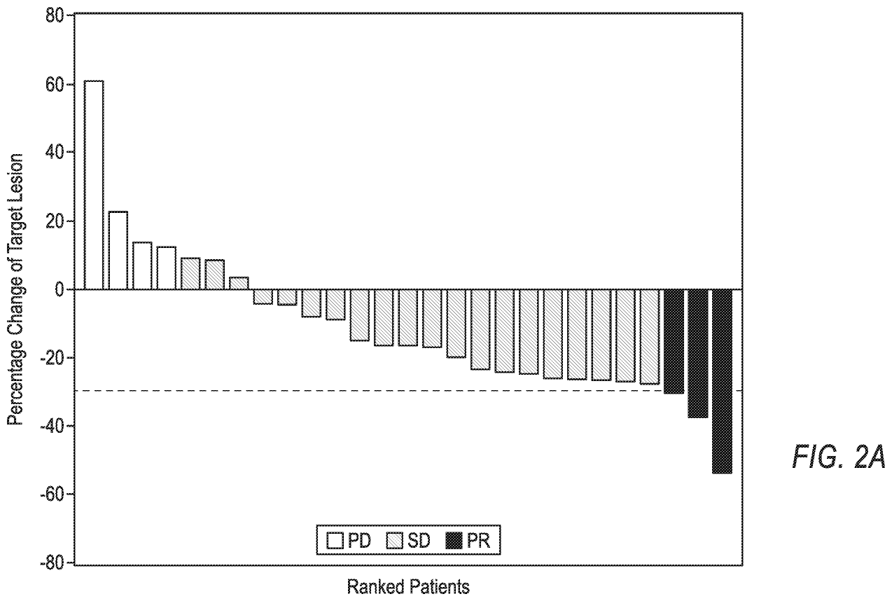

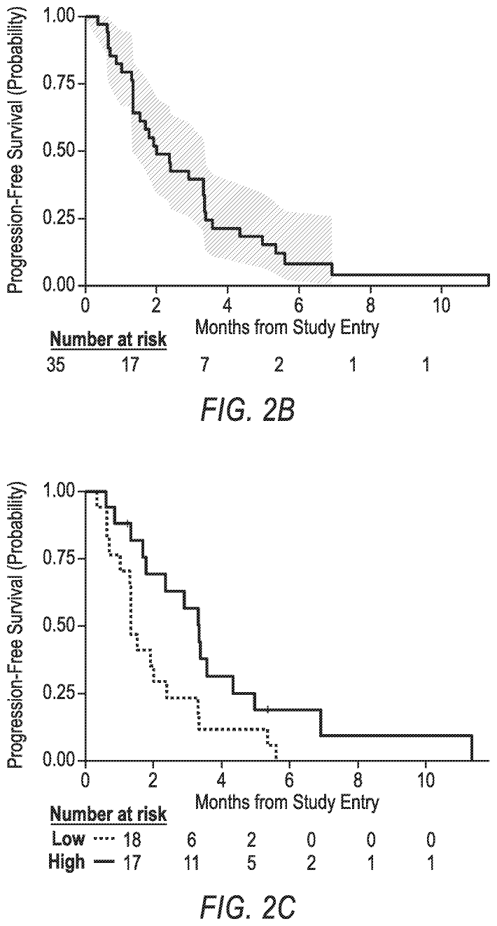

Efficacy: Patients received a median of 3 cycles (9 weeks) of therapy (range 1-17). One patient achieved a PR within the first 13 patients, so the study was continued to the second stage. A total of 3 patients achieved PR (ORR, 9% [95% CI: 2, 26]; Table 2 and FIG. 2A).

TABLE 2

| Best overall response | n (%) |
|---|---|
| PR | 3 (9) |
| SD | 20 (57) |
| ≥15 weeks | 9 (26) |
| <15 weeks | 11 (31) |
| PD^{a} | 11 (31) |
| Not evaluated due to toxicity | 1 (3) |

^{a}Including 7 patients(20%) with clinically progressive disease before protocol specified tumor assessment.
Abbreviations: PD, progressive disease; PR, partial response; RECIST, Response Evaluation Criteria in Solid Tumors; SD, stable diseases.

Thus, the study did not reach the level of clinical activity to define success under the Simon 2-stage design. Of these patients, one received 17 cycles of protocol therapy and was on treatment for 11.7 months, and another received 8 cycles of protocol therapy and was on treatment for 6.5 months. Twenty of 35 patients (57%) had SD as their best response, and 9 of 35 (26%) patients had SD for >15 weeks. The clinical benefit rate at 15 weeks was 34% 95% CI: 19%, 52% 1, and the median PFS was 2.0 months [1.3, 3.3] (FIG. 2B).

Twenty-one of 24 patients who reported pain upon entering the study completed at least one pain survey at week 1 or 4. Eleven (52%) of them reported a decrease in pain since baseline, and 10 of these had discontinued using pain medications.

Toxicity: The most common toxicities (all grades that were possibly related to protocol therapy) were fatigue (77%), diarrhea (40%), oral mucositis (37%), and palmar-plantar erythrodysesthesia (PPE; 37%; Table 3). There were 15 grade 3 adverse events, including elevated aspartate aminotransferase (n 5 2), elevated lipase (n 5 3), or hypertension (n 5 2). There were no grade 4 toxicities. Twelve patients (34%) required dose reduction, 4due to PPE and 8due to other toxicities. All but one patient omitted at least one dose while on protocol therapy, 26 due to toxicity and 8 due to other reasons. Overall, 32 patients (91%) went off treatment due to progressive disease and 3 (9%) due to toxicity.

TABLE 3

| | Maximum grade | | | |
|---|---|---|---|---|
| Adverse event | Total (% of 35) | Mild | Moderate | Severe |
| Fatigue | 27 (77) | 18 | 9 | 0 |
| Diarrhea | 14 (40) | 8 | 6 | 0 |
| Oral mucositis | 13 (37) | 11 | 2 | 0 |
| PPE | 13 (37) | 3 | 9 | 1 |
| Anorexia | 12 (34) | 10 | 2 | 0 |
| Elevated aspartate aminotransferase | 12 (34) | 7 | 3 | 2 |
| Hypertension | 12 (34) | 6 | 4 | 2 |
| Nausea | 10 (29) | 10 | 0 | 0 |
| Elevated alanine aminotransferase | 7 (20) | 6 | 0 | 1 |
| Dysgeusia | 7 (20) | 5 | 2 | 0 |
| Elavated lipase | 3 (9) | 0 | 0 | 3 |
| Prolonged activated partial thromboplastin time | 1 (3) | 0 | 0 | 1 |
| Bone pain | 1 (3) | 0 | 0 | 1 |
| Hypophosphatemia | 1 (3) | 0 | 0 | 1 |
| Infection | 1 (3) | 0 | 0 | 1 |
| Thromboembolic event | 1 (3) | 0 | 0 | 1 |
| Wound dehiscence | 1 (3) | 0 | 0 | 1 |

Abbreviation: PPE, palmar-plantar erythrodysesthesia

MET Amplification and Expression: MET Amplification and Expression Archival tissue analysis showed MET amplification in 2 of 35patients (MET/CEP7 2.14 and 2.16), and relative MET amplification (MET/CEP7 1.7) in 1 patient. These 3 patients were also the only ones to show relative MET gain in CTCs.

Plasma Biomarkers: Cabozantinib treatment was associated with an increase in plasma PIGF, VEGF, and VEGF-D from baseline to day 22, which was maintained at day 64 (p<0.001). Plasma CAIX also increased and sVEGFR2 decreased at days 43 and 64 (p, 0.001). Plasma HGF initially decreased at day 8, and then increased at day 64 (p5.02), whereas plasma SDF1a transiently increased at day 22 (p5.002) (Table 4). Plasma sVEGFR1, sMET, sTIE-2, or bFGF did not significantly change over time (Table 4). The kinetics of VEGF-C, GM-CSF, IL-1b, IL-2, IFN-g, IL-6, IL-8, IL-10, TNF-a, and IL-12/p70 were not analyzed because of the large number of undetectable measurements.

TABLE 4

| | Day 1 | | Day 8 | | Day 22 | | Day 43 | | Day 64 | | |
|---|---|---|---|---|---|---|---|---|---|---|---|
| Biomarker | n | MEdian (IQR) | n | Median (IQR) | n | Median (IQR) | n | Median (IQR) | n | Median (IQR) | p value* |
| [symbol] | 35 | 1.319 | 33 | 1.078 | 29 | 1.132 | 22 | 1.191 | 17 | 1.280 | .01 |
| | | (1.095-1.818) | | (954-1.485) | | (988-1.718) | | (1.051-1.324) | | (1.039-1.470) | |
| sMET | 35 | 795 | 33 | 890 | 29 | 902 | 22 | 903 | 17 | 923 | .45 |
| | | (678-1.954) | | (761-987) | | (736-1.005) | | (740-1.074) | | (822-1.104) | |
| [symbol] | 34 | 111 | 31 | 132 | 26 | 162 | 21 | 215 | 17 | 264 | <.001 |
| | | (58-205) | | (77-290) | | (116-283) | | (143-355) | | (153-413) | |
| [symbol] | 35 | 2.017 | 33 | 2.232 | 29 | 2.264 | 22 | 2.130 | 17 | 2.215 | .002 |
| | | (1.742-2.258) | | (1.765-2.326) | | (2.056-2.443) | | (1.940-2.355) | | (2.022-2.373) | |
| VEGF R2 | 35 | 8.872 | 33 | 8.475 | 25 | 6.726 | 22 | 5.812 | 17 | 5.968 | <.001 |
| | | (8.305-10.545) | | (7.271-5.725) | | (5.518-7.360) | | (4.051-6.781) | | (5.578-6.705) | |
| bFGF | 35 | 39 | 33 | 41 | 29 | 28 | 22 | 33 | 17 | 21 | .15 |
| | | (19-56) | | (29-53) | | (17-46) | | (24-49) | | (15-34) | |
| [symbol] | 35 | 53 | 33 | 89 | 29 | 119 | 22 | 124 | 17 | 119 | <.001 |
| | | (44-69) | | (79-140) | | (94-184) | | (82-162) | | (105-150) | |
| sPLT-3 | 35 | 1.24 | 33 | 50 | 29 | 90 | 22 | 124 | 17 | 87 | .39 |
| | | (.82-3.10) | | (63-180) | | (65-232) | | (69-245) | | (72-168) | |
| [symbol] | 35 | 4.648 | 33 | 5.038 | 29 | 4.845 | 22 | 4.636 | 17 | 5.256 | .06 |
| | | (3.932-5.627) | | (4.303-5.724) | | (4.368-5.638) | | (4.384-5.576) | | (4.546-5.472) | |
| VEGF | 35 | 98 | 32 | 188 | 26 | 206 | 23 | 206 | 17 | [symbol] | <.001 |
| | | (71-143) | | (126-316) | | (167-410) | | (125-342) | | (173-221) | |
| VEGF-D | 35 | 1.062 | 33 | 1.419 | 29 | 1.582 | 22 | 1.437 | 17 | 1.429 | <.001 |
| | | (.748-1.257) | | (1.102-1.806) | | (1.365-2.018) | | (1.035-1870) | | (1.121-2.063) | |

Median and IQR for VEGF-C, GM-CSF, IL-1β, IL-2, IFN-γ, IL-6, IL-8, IL-10, TNF-α, and IL-12/p70 were not tabulated because the majority of them had median values under the detectable threshold.
*p values were from mixed effects linear model, adjusted for multiple comparison using fake-discovery rate method.
Abbreviations: bFGF, basic fibroblast growth factor; CAIX, carbonic anhydrase IX; GM-CSF, granulocyte-macrophage colony stimulating factor; HGF, hepatocyte growth factor; IFN-γ, interferon-γ; IL-1β, interleukin 1β; IQR, interquartile range; PIGF, placental growth factor; SDF1α, stromal cell-derived factor 1α; sFLT-1, soluble fms-like tyrosinekinase 1; sMET, soluble MET; TNF-α, tumor necrosis factor α; VEGF, vascular endothelial growth factor; VEGFR2, vascular endothelial growth factor receptor 2. [symbol]
indicates data missing or illegible when filed

Of all biomarkers analyzed at baseline, only high baseline sMET (≥795 ng/mL median value) was associated with prolonged PFS (median PFS 3.3 months, lower 95% confidence limit 2.4), compared with low sMET(<795 ng/mL, median PFS 1.3 [1.3,3.3] months, p 5 .03) (FIG. 2C). There was a nonsignificant trend toward greater baseline sMET in patients with clinical benefit (1,008 pg/mL [interquartile range (IQR): 858, 1089] compared with those who did not (759 pg/mL [IQR: 663, 921]) (unadjusted p=0.06). The changes in plasma VEGF-C at day 22 correlated with clinical benefit (p5.03), but only samples from 19 of 35 patients were available at this time-point.

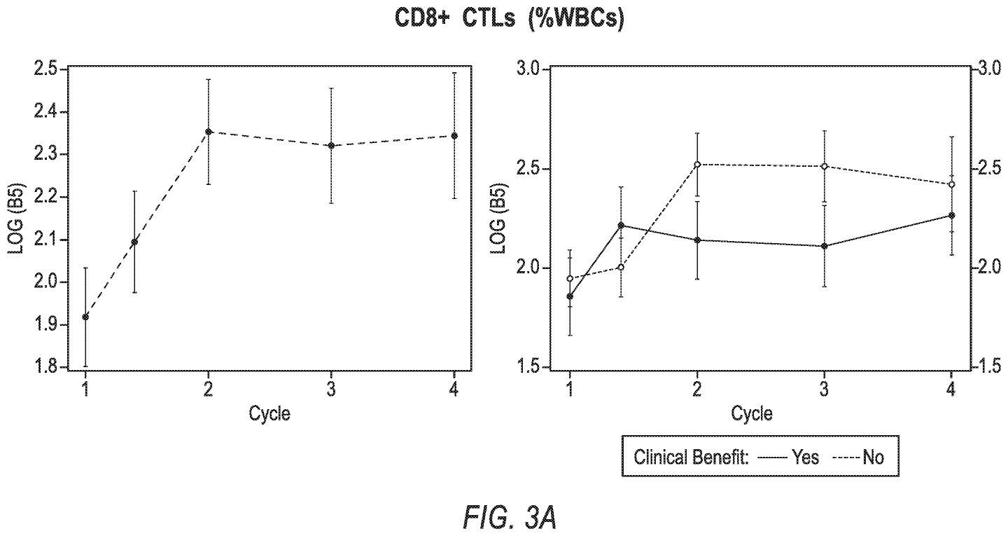

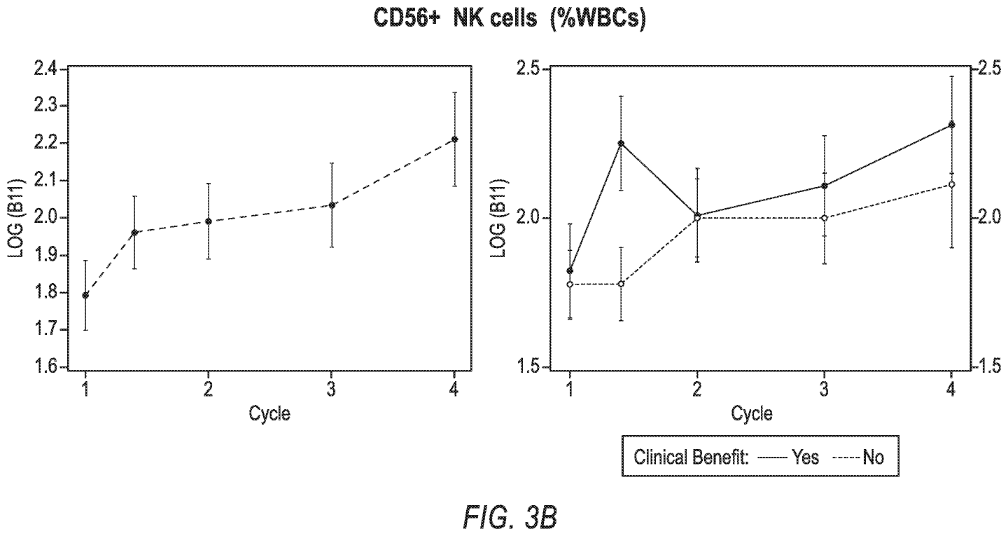

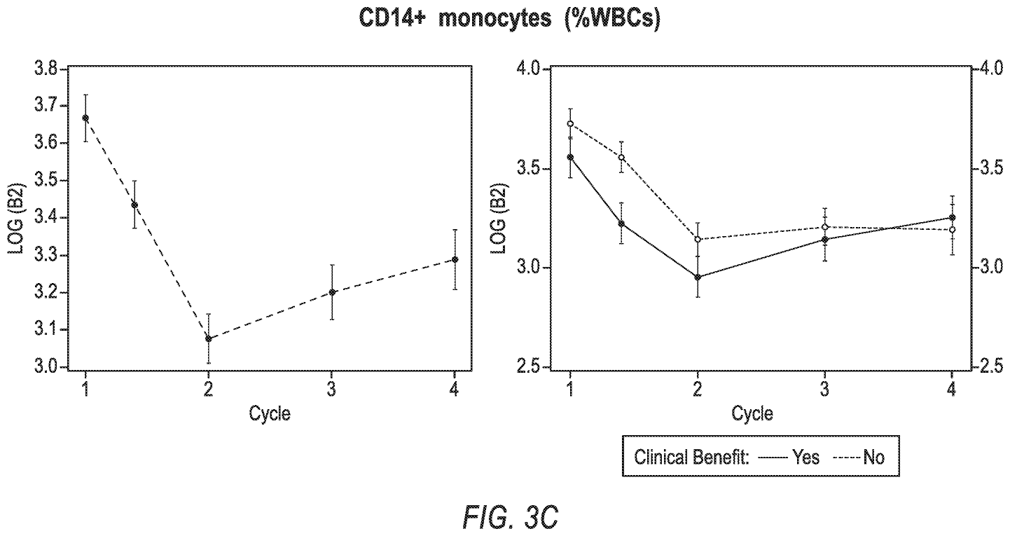

Cell Biomarkers: After cabozantinib treatment, we detected a significant increase in the fraction of circulating CD31 cells and CD31 CD4-CD81 T lymphocytes at days 22 and 64 (p=0.04 and p=0.01, respectively), and a decrease in percentage of CD141 monocytes at days 22 and 64 (p 5 .01) (Table 5). There was a nonsignificant trend toward increase in CD3+CD4+CD8−T (p=0.008) and CD3−CD561 NK lymphocytes (p=0.07), but changes in the fractions of CD1331 progenitor/stem cells, CD4+CD25+ regulatory T cells, CD4+CD127+ memory T cells, or CD3+CD56+ NKT cells (FIGS. 3A-3C and Table 5). None of the cell biomarkers associated with outcome measures.

TABLE 5

| Biomarker | Day 1 | | Day 8 | | Day 22 | | Day 43 | | Day 64 | | |
|---|---|---|---|---|---|---|---|---|---|---|---|
| % of WBC | n | Median (IQR) | n | Median (IQR) | n | Median (IQR) | n | Median (IQR) | n | MEdian (IQR) | p value* |
| CD34 + CD133+ | 34 | 0.17 | 32 | 0.11 | 28 | 0.12 | 19 | 0.18 | 16 | 0.11 | .83 |
| ( [symbol] cells) | | (0.07-0.37) | | (0.05-0.27) | | (0.05-0.32) | | (0.08-0.31) | | (0.06-0.29) | |
| CD14+ | 33 | 42.20 | 32 | 31.44 | 28 | 19.03 | 20 | 24.79 | 15 | 23.48 | .01 |
| (monocytes) | | (34.67-45.98) | | (23.83-41.12) | | (15.25-27.46) | | (16.37-31.30) | | (17.40-32.42) | |
| CD117 [symbol] + | 33 | 0.47 | 32 | 0.45 | 28 | 0.40 | 20 | 0.47 | 15 | 0.53 | .72 |
| | | (0.30-0.74) | | (0. [symbol] -0.85) | | (0.28-1.09) | | (0.35-0.62) | | (0.29-0.84) | |
| CD3+ | 34 | 34.73 | 31 | 24.06 | 28 | 33.56 | 21 | 32.79 | 16 | 30.99 | .04 |
| (lymphocytes) | | (13.45-29.95) | | (17.72-32.54) | | (23.45-45.58) | | (24.86-42.88) | | (19.45-43.33 | |
| CD3 + CD4 − CD8 [symbol] | 34 | 7.83 | 31 | 8.36 | 28 | 11.99 | 21 | 11.44 | 16 | 11.32 | .01 |
| (CTLs) | | (4.38-11.41) | | ( [symbol] -13.05) | | (7.71-16.88) | | (6.75-16.82) | | (7.28-14.33) | |
| CD3 + CD8 − CD4+ | 34 | 0.15 | 31 | 0.17 | 28 | 0.18 | 21 | 0.19 | 16 | 0.19 | .08 |
| | | (0.03-0.19) | | ( [symbol] -0.19) | | (0.14-0.25) | | (0.17-0.26) | | (0.12-0.30) | |
| CD3 + CD8 − CD4 + CD25+ | 34 | 0.66 | 31 | 0.79 | 28 | 0.59 | 21 | 0.73 | 16 | 0.61 | .98 |
| | | (0.26-1.53) | | (0.22-1.34) | | (0.35-2.24) | | (0.15-1.06) | | (0.36-1.61) | |
| CD3 + CD8 − CD4 + | 34 | 0.62 | 31 | 0.68 | 28 | 0.55 | 21 | 0.71 | 16 | 0.61 | .83 |
| CD25 + CD12 [symbol] − | | (0.26-1.30) | | (0.20-1.01) | | (0.32-1.95) | | (0.15-1.03) | | (0.35-1.45) | |
| ( [symbol] ) | | | | | | | | | | | |
| CD3 + CD8 − CD4 + CD25− | 34 | 11.41 | 31 | 14.18 | 28 | 16.41 | 21 | 17.71 | 16 | 15.29 | .06 |
| | | (7.57-14.62) | | (7.06-12.74) | | (12.41-23.24) | | (11.45-23.02) | | (9.67-25.14) | |
| CD4 + CD25 − CD117+ | 34 | 0.32 | 31 | 0.46 | 28 | 0.32 | 21 | 0.45 | 16 | 0.40 | .72 |
| ( [symbol] cells) | | (0.05-0.98) | | (0.13-1.08) | | (0.08-0.87) | | (0.16-1.06) | | (0.30-1.29) | |
| CD3 − CD56+ | 34 | 5.84 | 31 | 7.27 | 28 | 8.51 | 21 | 6.50 | 16 | 8.13 | .07 |
| (NK cells) | | (4.86-9.32) | | (4.58-9.44) | | (4.25-13.49) | | (5.21-9.80) | | (6.39-15.98) | |
| CD3 + CD56+ | 34 | 0.77 | 31 | 0.85 | 28 | 0.54 | 21 | 1.02 | 16 | 0.98 | .48 |
| (NKT cells) | | (0.35-1.83) | | (0.45-3.06) | | (0.43-3.17) | | (0.52-2.13) | | (0.71-1.04) | |

*p values were from mixed effects linear model and adjusted for multiple comparison using false-discovery rate method.
Abbreviations: CTLs. cytotoxic T lymphocytes; IQR. interquartile range; NK cells. natural killer cells; NKT cells. natural killer T cells; Trags. regulatory T cells; WBC. white blood cells. [symbol]
indicates data missing or illegible when filed

### Discussion

Cabozantinib monotherapy did not meet the pre-specified efficacy endpoint (ORR was 9%), but showed a clinical benefit rate of 34% at 15 weeks, and a median PFS of 2.0 months in Pretreated mTNBC patients. Treatment was well tolerated, and most common grade 3 toxicities were fatigue, diarrhea, oral mucositis, and PPE. Patients often reported decreases in pain, with some able to discontinue analgesics, consistent with previous results showing improvements in pain and reduction in narcotic use after cabozantinib.

MET remains an attractive target in TNBC, as shown in recent preclinical studies. Two patients enrolled in this study (6%) had tumors with MET amplification (consistent between archival tumor specimen and CTC evaluations), one of who discontinued therapy due to toxicity. Thus, no potential correlation could be established between MET amplification and response. However, high baseline plasma concentrations of sMET were associated with longer PFS, indicating that cancers producing increased sMET may be more likely to respond to MET inhibition. Larger randomized studies should validate the association of sMET with outcomes (OS, PFS, or pain) and to establish whether sMET is a prognostic or predictive in TNBC. The concentration of plasma HGF, the MET ligand, was lower in patients with clinical benefit versus those without, but this association did not reach statistical significance. Further larger studies examining the association of MET amplification in the tumor and circulating HGF with response to MET inhibition in TNBC are warranted.

Cabozantinib treatment was associated with changes in biomarker concentrations that are consistent with antivascular effects and increases in tissue hypoxia—increases in plasma CAIX, PIGF, VEGF, VEGF-D, and SDF1a. Moreover, cabozantinib significantly decreased plasma concentrations of sVEGFR2, a potential “pharmacodynamic” biomarker for anti-VEGFR2 TKIs. None of these systemic changes were associated with clinical outcomes. An increase in plasma VEGF-C associated with lack of clinical benefit and is worthy of further investigation.

Flow-cytometric analyses showed a persistent increase in the fraction of circulating CD31 T cells after cabozantinib therapy, largely driven by the increased CD4/CD8+ cytotoxic T lymphocyte (CTL) population. Moreover, there was a persistent decrease in the CD14+ monocytes, a mixed population that encompasses immunosuppressive and proangiogenic myeloid cells. These findings may reflect an activation of systemic antitumor immunity after treatment with cabozantinib, as observed in preclinical models, but did not associate with outcome. These findings are provocative given recent interest in combining cabozantinib with immune checkpoint inhibitors (NCT02496208).

The mechanism of action and of clinical benefit of VEGFR and MET inhibitors, when used alone or in combination, remains unclear. Several VEGF and MET inhibitors have been previously shown to be ineffective in metastatic breast cancer. The mechanism of benefit to VEGF blockade may be related to vascular normalization rather than antivascular effects and inducing hypoxia in the tumors. HGF and MET are hypoxia-inducible proteins, and increased MET expression after VEGFR2 inhibition has been associated with evasive treatment resistance. Unfortunately, antibody blockade of both VEGF using bevacizumab and MET using onartuzumab with paclitaxel demonstrated no clinical benefit in patients with mTNBC who had not previously received paclitaxel for metastatic disease. Our circulating biomarker data indicate that cabozantinib might have potent antivascular effects in mTNBC. To overcome these limitations, our hypothesis generating results indicate that: (a) sMET should be further studied as a potential biomarker of response; and (b) the systemic changes in antitumor immunity may be leveraged by rational combinations with immunotherapies.

This study has several limitations, related to the single-arm design and small number of patients. Clinically, the median PFS was modest, largely driven by the early PD in the patients without benefit. Future studies (such as NCT01441947 (cabozantinib with fulvestrant) and NCT0226053 (cabozantinib with trastuzumab) are warranted and should validate the biomarker data and characterize the tumors in the patients who benefit from therapy.

This phase II study of cabozantinib showed an ORR of 9%, preliminary activity, and favorable safety in mTNBC patients. Exploratory analyses showed that circulating sMET levels may be potentially a response biomarker for cabozantinib and that this agent may have an intriguing immunomodulatory activity. These hypotheses should be tested in larger studies in mTNBC and other malignancies.

### Other Embodiments

The foregoing disclosure has been described in some detail by way of illustration and example, for purposes of clarity and understanding. The invention has been described with reference to various specific and preferred embodiments and techniques. However, it should be understood that many variations and modifications can be made while remaining within the spirit and scope of the invention. It will be obvious to one of skill in the art that changes and modifications can be practiced within the scope of the appended claims. Therefore, it is to be understood that the above description is intended to be illustrative and not restrictive. The scope of the invention should, therefore, be determined not with reference to the above description, but should instead be determined with reference to the following appended claims, along with the full scope of equivalents to which such claims are entitled.

1-32. (canceled)

33. A method of treating triple negative breast cancer in a human patient, comprising administering to a patient in need of such treatment cabozantinib or a pharmaceutically acceptable salt thereof at a dose which activates circulating cell biomarkers.

34. The method of claim 33, wherein the circulating cell biomarkers are one or more circulating biomarkers of the immune system.

35. The method of claim 34, wherein the one or more circulating biomarkers is selected from the group consisting of CD31 cells, CD31 CD4-CD81 T lymphocytes, CD141 monocytes, CD3+CD4+CD8-T lymphocytes, CD3-CD561 NK lymphocytes, CD1331 progenitor/stem cells, CD4+CD25+ regulatory T cells, CD4+CD127+ memory T cells, and CD3+CD56+ NKT cells.

36. The method of claim 33, wherein circulating cell biomarker activation is determined by measuring at least one circulating cell biomarker expressed by the patient.

37. The method of claim 33, wherein the circulating cell biomarker is selected from the group consisting of CD3+ cells, CD8+ T cells, CD4+ cells, CD56+NK cells, and CD14+cells.

38. The method of claim 33, wherein cabozantinib is administered as cabozantinib (S)-malate.

39. The method of claim 38, wherein the cabozantinib (S)-malate is administered as a tablet formulation comprising approximately (% w/w):
30-32 percent by weight of cabozantinib, (S)-malate salt;
38-40 percent by weight of microcrystalline cellulose;
18-22 percent by weight of lactose;
2-4 percent by weight of hydroxypropyl cellulose;
4-8 percent by weight of croscarmellose sodium;
0.2-0.6 percent by weight of colloidal silicon dioxide;
0.5-1 percent by weight of magnesium stearate; and further comprising:
a film coating material comprising hypromellose, titanium dioxide, triacetin, and iron oxide yellow,
or wherein the cabozantinib (S)-malate is administered as a tablet formulation comprising approximately (%w/w):
31-32 percent by weight of cabozantinib, (S)-malate salt;
39-40 percent by weight of microcrystalline cellulose;
19-20 percent by weight of lactose;
2.5-3.5 percent by weight of hydroxypropyl cellulose;
5.5-6.5 percent by weight of croscarmellose sodium;
0.25-0.35 percent by weight of colloidal silicon dioxide;
0.7-0.8 percent by weight of magnesium stearate; and further comprising:
3.9-4.1 percent by weight of a film coating material comprising hypromellose, titanium dioxide, triacetin, and iron oxide yellow.

40. The method of claim 38, wherein cabozantinib (S)-malate is administered as a tablet formulation containing 20, 40, or 60 mg of cabozantinib FBE.

41. The method of claim 38, wherein cabozantinib (S)-malate is administered as a tablet formulation selected from the group consisting of:

| | Theoretical Quantity (mg/unit dose) | | |
|---|---|---|---|
| Ingredient | 20-mg Tablet* | 40-mg Tablet* | 60-mg Tablet* |
| Cabozantinib (S)-malate | 25.34 | 50.69 | 76.03 |
| Microcrystalline Cellulose, PH-102 | 31.08 | 62.16 | 93.24 |
| Lactose Anhydrous, 60M | 15.54 | 31.07 | 46.61 |
| Hydroxypropyl Cellulose, EXF | 2.400 | 4.800 | 7.200 |
| Croscarmellose Sodium | 4.800 | 9.600 | 14.40 |
| Colloidal Silicon Dioxide | 0.2400 | 0.4800 | 0.7200 |
| Magnesium Stearate (Non-Bovine) | 0.6000 | 1.200 | 1.800 |
| Opadry ® Yellow (03K92254) | 3.200 | 6.400 | 9.600 |
| Total tablet weight | 83.20 | 166.4 | 249.6 |

*Free Base Equivalent (FBE)

42. The method of claim 38, wherein the cabozantinib (S)-malate is administered once daily.

43. The method of claim 33, wherein the amount of cabozantinib that is administered once daily is 60 mg FBE.

44. A method of treating triple negative breast cancer in a human patient, comprising administering to a patient in need of such treatment cabozantinib or a pharmaceutically acceptable salt thereof at a dose which activates circulating cell biomarkers, in combination with one or more additional therapies or agents.

45. The method of claim 44, wherein triple negative breast cancer is HER2 triple negative breast cancer.

46. The method of claim 45, wherein the one or more circulating biomarkers is selected from the group consisting of CD31 cells, CD31 CD4-CD81 T lymphocytes, CD141 monocytes, CD3+CD4+CD8-T lymphocytes, CD3-CD561 NK lymphocytes, CD1331 progenitor/stem cells, CD4+CD25+ regulatory T cells, CD4+CD127+ memory T cells, and CD3+CD56+ NKT cells.

47. The method of claim 45, wherein the HER2 triple negative breast cancer is HER3+ or FISH-positive breast cancer.

48. The method of claim 45, wherein the one or more additional agents is an immune modulator selected from the group consisting of trastuzumab, pertuzumab, ado-trastuzumab emantine, lapatinib, fulvestrant, pemborlizumab, nivolumab, ipilimumab, durvalumab, tremelimumab, epacadostat, atezolizumab, and PDR001.

49. The method of claim 44, wherein the one or more circulating biomarkers is selected from the group consisting of CD31 cells, CD31 CD4-CD81 T lymphocytes, CD141 monocytes, CD3+CD4+CD8-T lymphocytes, CD3-CD561 NK lymphocytes, CD1331 progenitor/stem cells, CD4+CD25+ regulatory T cells, CD4+CD127+ memory T cells, and CD3+CD56+ NKT cells.

50. The method of claim 44, wherein the one or more additional agents is selected from the group consisting of trastuzumab, pertuzumab, ado-trastuzumab emantine, and lapatinib.

51. The method of claim 44, wherein the one or more additional agents is a vaccine, wherein the vaccine is selected from the group consisting of nelipepimut-S, INO-1400, INO-9012, OBI-833, MAG-Tn3 HER-2 peptide vaccine, a personalized vaccine, and POLY-ICLC.

52. The method of claim 44, wherein the one or more additional agents is selected from the group consisting of the LAG fusion protein IMP321, the anti-OX40 antibody MEDI6469, and the B7-H3×CD3 DART protein MGD009.

53. The method of claim 44, wherein the one or more additional therapy is selected from the group consisting of adoptive T-cell transfer, oncolytic virus therapy, antibodies, adjuvant immunotherapies, and cytokines.

54. A method of treating triple negative breast cancer in a human patient having a baseline plasma concentration of sMET that is greater than the median baseline plasma concentration of sMET in humans, comprising administering to the patient an amount of cabozantinib or a pharmaceutically acceptable salt thereof, wherein the amount of cabozantinib is sufficient to activate the immune system.

55. The method of claim 54, wherein the baseline plasma concentration of sMET greater than or equal to 795 mg/mL median value.

56. The method of claim 55, wherein progression free survival of patients having a baseline plasma concentration of sMET of greater than or equal to 795 mg/mL median value is extended as compared to patients having a baseline plasma concentration of sMET of less than 795 mg/mL median value.
